(19) United States
(12) Patent Application Publication
(10) Pub. No.: US 2014/0001282 A1
(43) Pub. Date: Jan. 2, 2014

(54) Aroma Specialty Plate

(71) Applicant: Wanda Battle-Frazier, Plainfield, NJ (US)

(72) Inventor: Wanda Battle-Frazier, Plainfield, NJ (US)

(21) Appl. No.: 13/887,974

(22) Filed: May 6, 2013

Related U.S. Application Data
(60) Provisional application No. 61/688,518, filed on May 16, 2012.

(51) Int. Cl. A61L 9/03

(52) U.S. Cl. CPC A61L 9/03
USPC 239/58; 239/34

(57) ABSTRACT

The invention provides ceramic adornments for dessert dishes that are infused with a temperature activated scent to match the food item placed on the plate. Aromatic adornments attach directly to the surface of a companion plate and include a wide variety of fruit based shapes such as strawberry, lemon, apple and orange, and each ceramic piece is infused with a scent germane to the fruit it depicts. To facilitate the release of the aroma, each piece is outfitted with a series of vent openings.

### CLAIM OF PRIORITY

This patent application claims priority under 35 USC 119 (e) (1) from U.S. Provisional Patent Application Ser. No. 61/688,518 filed May 16, 2012, of common inventorship herewith entitled, “Aroma Specialty Plate.”

### FIELD OF THE INVENTION

The present invention pertains to the field of food enhancement and more specifically to the field of food aroma releasing methods.

### BACKGROUND OF THE INVENTION

The prior art has put forth several designs for food aroma releasing methods. Among these are:

U.S. Pat. No. 5,071,704 to Fariba Fischel-Ghodsian describes a controlled release device useful for the release of vapors or liquids. The device is a multilayered laminate consisting of a reservoir layer which incorporates an active compound such as a perfume or fragrance or insect repellant, an impermeable membrane layer adjacent the reservoir layer, and a diffusion rate limiting membrane layer adjacent the reservoir layer. The device preferably includes an adhesive layer for adhering the device to skin or a surface and an ornamental decorative layer.

U.S. Pat. No. 6,066,347 to Nikhil Prasad and Jeffrey Willey describes a food package having a built in aroma. The package includes a food compartment and an aromatized substrate comprising a heat sensitive sealant which captures the aroma. The aromatized substrate is effective to preserve the aroma during freezing, refrigeration and ambient storage, and is capable of releasing the aroma when the package with the food in the food compartment is heated in a microwave oven.

U.S. Pat. No. 7,741,266 to Adam Bell, Mohamed Mohamed, Robert Zwick and James Bumenfeld describes a process for optimizing the fragrance of a scented cellulosic.

None of these prior art references describe the present invention.

### SUMMARY OF THE INVENTION

It is an object of the present invention to provide ceramic adornments for a serving plate that emanates a scent similar to the scent of the food being served.

### BRIEF DESCRIPTION OF THE DRAWINGS

FIG. 1 is a prototypical view showing aromatic adornments in the form of strawberries.

FIG. 2 is a prototypical view showing aromatic adornments in the form of apples.

FIG. 3 is a prototypical view showing aromatic adornments in the form of cherries.

FIG. 4 shows a series of top perspective views of the above aromatic adornments attached to the top of a plate.

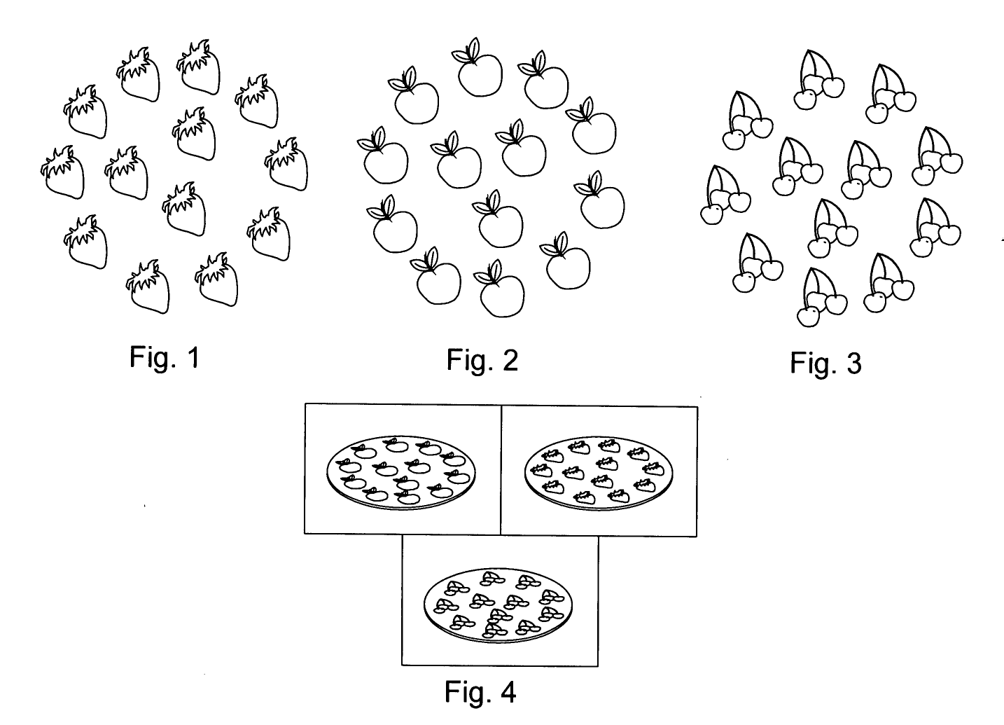

### DETAILED DESCRIPTION OF THE INVENTION

Inventions are always a product of vision and creativity, whether they are world changing inventions like cars or computers or everyday conveniences like pump hairsprays, cordless tools or resealable soda bottles. Some inventions present an entirely new product like a cell phone and such an invention changes the way we live. Other inventions such as a snow scraper and brush combine two existing tools or products into one tool, reduce clutter in one's environment and make one's chores easier to perform. Some inventions deal with common problems such as roll on deodorants dealing with body odors and other inventions like the automobile bring a new form of convenience when they come into existence. Many inventions consist of relatively small but highly effective improvements of existing products or technologies. Inventions come in so many categories, and sometimes necessity or sometimes creativity is the mother of invention. Somewhere, someone is combining their own vision, imagination and creativity to create something that will improve, or even revolutionize, our lives. Most inventions fill a relatively small need or perform a specialized and limited function. If that need or functionality is shared by a sufficient number of consumers, the invention has a great potential for success.

The present invention, hereinafter referred to as the Aroma Specialty Plate, is a line of serving plates and specially designed ceramic adornments for dessert dishes that are infused with a temperature activated scent to match the food item placed on the plate. Design intent is to provide a refreshing and delectable enhancement to standard dinner ware. The durable ceramic and porcelain. Aroma Specialty Plate available in this line measures approximately six to eight inches in diameter. Unique to this product and what sets it apart from the standard dessert dish is found in its decorative aromatic accoutrements. These components are offered as a selection of ceramic and porcelain pieces designed to attach directly to the surface of a companion plate. In one embodiment of the present invention, these accoutrements are placed on serving plates included in this dinnerware line. In another embodiment of the present invention, these accoutrements are available in sets of individual accoutrements which are placed on any serving plate such as paper, plastic, glass, stoneware, porcelain or ceramic. Made available in a wide variety of fruit based shapes such as strawberry, lemon, apple and orange, each ceramic piece is infused with a scent germane to the fruit it depicts. To facilitate the release of the aroma, each piece is outfitted with a series of vent openings. Hot and cold temperatures react positively with ceramic material and along with a dessert item placed onto a plate adorned with these decorations, the aromatic potential of the dessert being served is increased. When a slice of hot apple pie is served on an Aroma Specialty Plate featuring apples, a scrumptious scent of apple permeates the dining experience. A plate comprised of a selection of oranges emanates an orange's delectable tart aroma when serving orange sherbet, doubling the citrusy delight. Vents of the fruit based attachments are opened easily for cleaning. The Aroma Specialty Plate adornments may be washed by hand or in a dishwasher, then dried and stored in an airtight and waterproof container between uses.

Creative and unique, the Aroma Specialty Plate lends both aesthetic and fragrant appeal to a wide variety of dessert servings. Guests delight in receiving strawberry shortcake that is placed on a striking dish festooned with decorative strawberries and also fills the room with a wonderful strawberry aroma. Designed for home use, this versatile Aroma Specialty Plate proves a hit in the restaurant setting, adding that something extra to cap off an amazing meal. Affordably priced, this creative line of adornments for dessert plates are well received by home chefs as well as dining establishments.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. An aromatic adornment for a serving plate that emanates a scent that is the same scent as the food being served, comprising: an essentially circular ceramic or porcelain piece infused with a scent, wherein the circular piece attaches directly to the surface of a companion plate.

2. The aromatic adornment of claim 1, wherein the circular piece depicts a fruit selected from the group consisting of: strawberry, lemon, apple and orange; and wherein scent infusing the circular piece is selected from the group consisting of: strawberry, lemon, apple and orange, and wherein the fruit depicted matches the infused scent.

3. An aromatic adornment for a serving plate that emanates a scent that is the same scent as the food being served, comprising: a ceramic or porcelain piece infused with a scent, wherein the circular piece attaches directly to the surface of a companion plate, wherein the shape of the piece resembles the shape of a fruit selected from the group consisting of: strawberry, lemon, apple and orange.

4. The aromatic adornment of claim 1, further comprising vent openings in the piece to allow the egress of scent.

5. The aromatic adornment of claim 3, further comprising vent openings in the piece to allow the egress of scent.

6. The aromatic adornment of claim 4, wherein the vents are closable.

7. The aromatic adornment of claim 5, wherein the vents are closable.

8. The aromatic adornment of claim 4, wherein the adornment may be washed by hand or in a dishwasher, then dried and stored in an airtight and waterproof container between uses.

9. The aromatic adornment of claim 5, wherein the adornment may be washed by hand or in a dishwasher, then dried and stored in an airtight and waterproof container between uses.

10. The aromatic adornment of claim 1, wherein the companion plate measures approximately six to eight inches in diameter.

11. The aromatic adornment of claim 3, wherein the companion plate measures approximately six to eight inches in diameter.

12. The aromatic adornment of claim 1, wherein the scent is temperature-activated.

13. The aromatic adornment of claim 3, wherein the scent is temperature-activated.
